(19) United States
(12) Patent Application Publication
(10) Pub. No.: US 2006/0216355 A1
(43) Pub. Date: Sep. 28, 2006

(54) Encapsulated pharmaceuticals in a medium having non-encapsulated flavoring agents

(76) Inventor: Donald Spector, New York, NY (US)

Correspondence Address: Glen M. Diehl;NORTON & DIEHL LLC, Suite 110, 77 Brant Avenue, Clark, NJ, 07066, US

(21) Appl. No.: 11/131,141

(22) Filed: May 17, 2005

Related U.S. Application Data
(60) Provisional application No. 60/665,560, filed on Mar. 28, 2005.

(51) Int. Cl. B01J 13/04; A61K 9/50; A61K 9/16

(52) U.S. Cl. 424/490; 264/4.1

(57) ABSTRACT

A pharmacologically active ingredient is encapsulated, and in this encapsulated form, is dispersed throughout a pleasantly flavored medium. When the medium is orally ingested, encapsulation of the pharmacologically active ingredient shields the recipient from any associated unpleasant taste, while beneficially providing the pleasant taste of the flavoring of the medium. The medium may be a liquid or a solid.

### BACKGROUND OF THE INVENTION

The present invention relates to the field of pharmaceuticals.

As many parents or pet owners can attest to, the administering of unpleasant-tasting medicines can be an arduous chore. To alleviate this problem, medicines are often combined with flavorings to mask the unpleasant taste of the pharmacologically active ingredients. Although such flavorings can help somewhat, they are ineffective in masking the taste of the prescribed medicine, and drugs using the flavorings still result in an acrid or bitter aftertaste. Young children and pets in particular find this unpleasant aftertaste objectionable.

Compounding this problem is the fact that the flavorings are encapsulated or coated. This encapsulation or coating helps to preserve the flavorings. A great many methods of micro-encapsulation are known in the art which provide for a quick release of the flavorings when ingested. Silica micro-encapsulation is one such technique. However, because of the micro-encapsulation, the unpleasantly tasting pharmacologically active compounds reach the tongue before the flavorings, interfering with the purpose of the flavorings, and thus increasing patient distress.

By way of example, U.S. Pat. No. 3,971,852 and U.S. Pat. No. 3,314,803 describe methods for encapsulating flavor compounds. Thus, both of these patents describe methods by which the flavor components are masked from patients, producing the undesirable effect of an unpleasant taste when a pharmacologically active agent is being administered.

U.S. Pat. No. 6,887,493 discloses an improved controlled release system that can be incorporated in oral care, food products, and beverages that can effectively encapsulate one or more types of active ingredients, flavors, or combinations of flavors and various active ingredients to release them one after the other in a consecutive manner or to provide change in flavor character. Again, the flavorings are being encapsulated, thereby masking their effectiveness and creating the previously described problems.

Improved methods for making administering and making such medicines as well as improved formulations of flavored medicines are therefore needed.

### BRIEF SUMMARY OF THE INVENTION

In accordance with one aspect of the present invention, a drug formulation comprises a medium, a flavoring agent dispersed throughout the medium, and a micro-encapsulated pharmaceutical dispersed throughout the medium. The medium may be either a liquid or a solid, or a suspension or a colloidal suspension. The encapsulated pharmaceutical comprises a first plurality of micro-capsules surrounding a pharmacologically active ingredient. The first plurality of micro-capsules should release the pharmacologically active ingredient some time after the drug is ingested.

In one aspect of the present invention, the flavoring agent is not encapsulated and is not coated. The flavoring agent is preferably dispersed throughout the medium, in a liquid, a suspension, a colloidal suspension or a solid. This allows maximum exposure of the flavoring agent to a patient upon administration of the drug.

It is a benefit of the present invention that encapsulation of the pharmacologically active ingredient shields the recipient from the unpleasant taste associated with the pharmacologically active ingredient. Simultaneously, the recipient receives the full benefit of the flavoring ingredient. Administration of the pharmacologically active ingredient is thus made less objectionable.

### BRIEF DESCRIPTION OF THE DRAWINGS

FIG. 1 illustrates a first aspect of a drug formulation in accordance with the present invention.

FIG. 2 illustrates another aspect of the present invention.

### DETAILED DESCRIPTION OF THE INVENTION

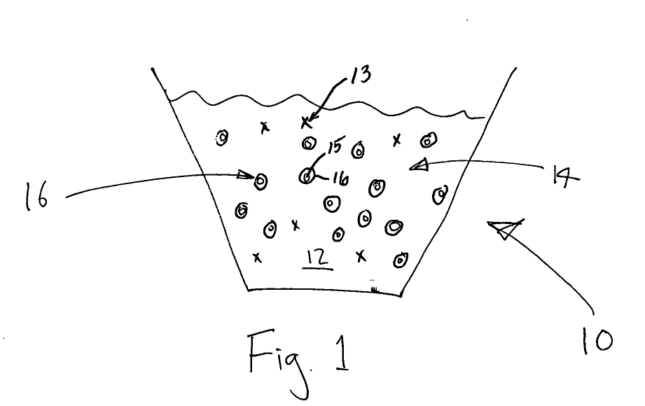

FIG. 1 illustrates a first aspect of the present invention. A drug formulation 10, comprises a liquid medium 12, a non-encapsulated flavoring agent 13 dispersed throughout the liquid medium 12, and a first plurality of micro-capsules 16 that encapsulate a pharmacologically active ingredient 15. The plurality of first micro-capsules 16 thus provide an encapsulated pharmaceutical that is dispersed throughout the liquid medium.

Methods of encapsulating in drug delivery systems are well known. Usually, however, it is the flavoring agent that is encapsulated. The sizing of the plurality of micro-capsules 16 will depend largely on the method of encapsulation used and on the item being encapsulated.

The liquid medium 12 may be of any suitable type known in the field of pharmacology, and may comprise, for example, water, alcohol, syrups or the like. Suitable candidates for the liquid medium 12 are well-known in the field, and their selection is within the means of one skill in the art. The medium 12 can also be a suspension or a colloidal suspension.

The non-encapsulated flavoring agent 13 may be any known flavoring agent, either natural or artificial. Preferably, the flavoring agent readily dissolves in, and is stable in, the liquid medium 12. Further, the flavoring agent should provide a pleasing flavor to the target recipient. Selection of suitable flavoring agents is also within the means of one skilled in the art. In accordance with one aspect of the present invention the non-encapsulated flavoring agent dispersed within the liquid medium 12 forms a flavored suspension 14 with the encapsulated pharmaceutical.

The plurality of micro-capsules 16 should be stable and relatively inert with respect to both the pharmacologically active ingredient 15 and the flavored suspension 14, i.e., the liquid matrix 12 and the non-encapsulated flavoring agent. By being minimally reactive with the liquid matrix 12, the micro-capsule 16 insures that the pharmacologically active ingredient 15, with its unpleasant taste, is not prematurely released into the liquid matrix 12. Minimal reactivity and stability may be determined, for example, by placing minimal acceptable limits on the percentage of micro-capsules 16 that prematurely release the pharmacologically active ingredient 15 into the liquid matrix 12 over the expected shelf-life of the drug delivery system 10.

Once ingested by the target recipient, the micro-capsule 16 dissolves or bursts, thereby releasing the pharmacologically active ingredient 15. Selection of suitable materials for the micro-capsule 16 is a skill well known to those in the art, as are the selection of the pharmacologically active ingredient 15 and the choosing of appropriate dosage levels for the pharmacologically active ingredient 15. One aspect of the micro-capsule 16 involves the release time, which is the amount of time that elapses after ingestion of the drug delivery system 10 before the micro-capsules 16 begin releasing the pharmacologically active ingredient 15. As is well known in the art, the release time can be varied as may be required. For example, the release time may be almost immediate, so that the pharmacologically active ingredient 15 is released while in the mouth. Alternatively, the release time may be delayed so that the pharmacologically active ingredient 15 is released only some time after being swallowed. Selection of an appropriate release time for the micro-capsules 16 is a skill familiar to those in the art. It may be desirable to set the release time of the micro-capsules 16 so that the liquid matrix 12 is swallowed before the pharmacologically active ingredient 15 is released, thus providing maximum protection to the recipient against the unpleasant taste of the pharmacologically active ingredient 15.

To provide the drug delivery system 10, a manufacturer selects a desired pharmacologically active ingredient 15. The manufacturer then selects a suitable material for the micro-capsule 16, and uses this material to perform an encapsulation process, which results in an encapsulated pharmaceutical; that is, a plurality of micro-capsules 16 that surround the pharmacologically active ingredient 15 having the desired release time. Numerous encapsulation processes exist, which utilize various types of materials. The manufacturer then selects a suitable liquid matrix 12 and non-encapsulated flavoring agent. The non-encapsulated flavoring agent is dispersed throughout the liquid matrix to form the flavored suspension 14. The micro-encapsulated pharmaceutical is then dispersed throughout the flavored suspension 14, at appropriate dosage levels, to provide the drug delivery system 10.

When orally administered to a patient, the micro-capsules 16 shield the patient from the taste of the pharmacologically active ingredient 15, while the flavored suspension 14 immediately provides a pleasing flavor. The drug delivery system and drug formulation 10 of FIG. 1 thus reduces unpleasant flavors and eases administration of the pharmacologically active ingredient 15.

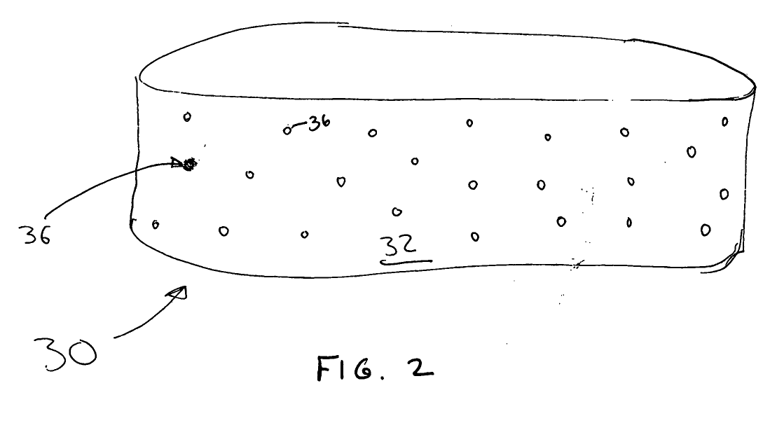

FIG. 2 illustrates another aspect of the present invention, a drug delivery system and a drug formulation 30. The drug 30 has a medium 32 that is a solid rather than a liquid. Selection of an appropriate material for the solid medium 32 is a well-known skill in the art. A non-encapsulated flavoring agent is dispersed through the solid medium 32 to provide flavor to the drug 30. The flavoring agent may be natural or artificial. An encapsulated pharmaceutical, in the form of micro-capsules 36 enclosing a pharmaceutical, is dispersed throughout the solid medium 32. Hence, when ingested, the recipient of the drug 30 first tastes the flavored solid medium 32, and is shielded by the plurality of micro-capsules 36 from the unpleasant flavor of the pharmacologically active ingredient contained within the plurality of micro-capsules 36.

The formulation is preferably manufactured by dispersing the flavoring agent in the medium. It is preferred that the flavoring agent be dispersed without encapsulation and with out coating throughout the medium. Then the active pharmaceutical agent is encapsulated by any of the well known encapsulation techniques and mixed with the medium. Accordingly, this process creates a drug formulation that masks the bad tasting active pharmaceutical agent from a patient while providing maximum exposure of the good tasting flavoring agents to the patient.

U.S. Pat. No. 3,971,852, U.S. Pat. No. 3,314,803 and U.S. Pat. No. 6,887,493 are incorporated by reference herein in their entirety.

Although the invention herein has been described with reference to particular embodiments, it is to be understood that these embodiments are merely illustrative of the principles and applications of the present invention. It will be apparent to those skilled in the art that various modifications and variations can be made in the apparatus and methods of the present invention without departing from the spirit and scope of the invention. Thus, it is intended that the present invention include modifications and variations that are within the scope of the appended claims and their equivalents.

1. A drug that can be orally administered, comprising:
a medium;
a flavoring agent dispersed in the medium, the flavoring agent not being encapsulated or coated; and
an encapsulated pharmaceutical dispersed in the medium.

2. The drug of claim 1, wherein the encapsulated pharmaceutical is adapted to be released after ingestion.

3. The drug of claim 1, wherein the encapsulated pharmaceutical comprises a first plurality of micro-capsules surrounding the pharmaceutical, and the first plurality of micro-capsules is stable and minimally reactive with the medium.

4. The drug of claim 1, wherein the medium is a liquid.

5. The drug of claim 1, wherein the medium is a solid.

6. The drug of claim 1, wherein the medium is a suspension.

7. The drug system of claim 1, wherein the medium is a colloidal suspension.

8. A method for reducing unpleasant taste in an orally administered drug, comprising administering a medium having an encapsulated pharmaceutical and a flavoring agent to a mammal, the flavoring agent not being encapsulated or coated, wherein upon the medium being administered, the mammal tastes the flavoring agent but does not taste the pharmaceutical.

9. A method of manufacturing a drug, comprising:
dispersing a non-encapsulated and non-coated flavoring agent throughout a medium;
encapsulating a pharmaceutical; and
dispersing the encapsulated pharmaceutical throughout the medium.

10. The method of claim 9, wherein the pharmaceutical is encapsulated with a first plurality of micro-capsules, the first plurality of micro-capsules being stable and minimally reactive with the medium.

11. The method of claim 9, wherein the medium is a liquid.

12. The method of claim 9, wherein the medium becomes a solid.

13. The method of claim 9, wherein the medium is a suspension.

14. The method of claim 9, wherein the medium is a colloidal suspension.
